(19) United States
(12) Patent Application Publication
(10) Pub. No.: US 2016/0250397 A1
(43) Pub. Date: Sep. 1, 2016

(54) ASPIRATION MAXIMIZING CATHETER

(71) Applicants: Stephan GRIFFIN, San Jose, CA (US); William CANNON, Galway (IE); John GILBERT, Galway (IE); Brendan MARRINAN, Galway (IE); Cormac CLARKE, Galway (IE); Richard GRIBBONS, Galway (IE); Bernard MCDERMOTT, Mayo (IE); Liam FARRISSEY, Galway (IE); CREGANNA-TACTX MEDICAL, Galway (IE)

(72) Inventors: Stephan Griffin, San Jose, CA (US); William Cannon, Galway (IE); John Gilbert, Galway (IE); Brendan Marrinan, Galway (IE); Cormac Clarke, Galway (IE); Richard Gribbons, Galway (IE); Bernard McDermott, Mayo (IE); Liam Farrissey, Galway (US)

(21) Appl. No.: 15/030,728

(22) PCT Filed: Oct. 28, 2014
(86) PCT No.: PCT/US14/62602

§ 371 Date: Apr. 20, 2016

Related U.S. Application Data
(60) Provisional application No. 61/897,508, filed on Oct. 30, 2013.

(51) Int. Cl. A61M 1/00; A61M 25/01

(52) U.S. Cl. CPC A61M 1/008; A61M 25/0172

(57) ABSTRACT

A catheter design and method is provided for the aspiration of thrombus, clot, atherosclerotic emboli in the vascular bed. The aspiration catheter includes an aspiration lumen, a guidewire lumen having an exchange port, and a recess in the aspiration unit. The aspiration catheter lumen is divided into two sections: (i) proximal, and (ii) distal. The distal section has a distal opening.

### BACKGROUND

### Field

This disclosure generally relates to medical devices and more particularly to catheters used for aspiration.

### SUMMARY

A catheter design and method is provided for aspiration, such as the aspiration of thrombus, clot, and/or atherosclerotic emboli in the vascular bed. The aspiration catheter includes an aspiration lumen, a guidewire lumen having an exchange port, and a recess in the aspiration lumen. The aspiration catheter lumen is divided into two sections: (i) proximal, and (ii) distal. The distal section has a distal opening.

The exchange port is configured between the distal segment and the proximal segment. In one implementation, the catheter comprises an elongated aspiration lumen defined by a selectively laser cut pattern from the proximal to distal end (or reinforced shaft), providing an optimal stiffness profile that permits the catheter to navigate tortuous anatomy. Over the distal portion (e.g., 10-20 cm) an axial recess is cut to permit the seating of the rapid exchange guidewire lumen, which is subsequently bonded to the aspiration lumen. The recess cut into the aspiration lumen improves the catheters efficient use of cross sectional area (CSA), thereby offering a maximum CSA for aspiration.

### BRIEF DESCRIPTION OF THE DRAWINGS

FIG. 1 illustrates a catheter having a guidewire lumen, consistent with an embodiment of the present disclosure.

FIG. 2 illustrates a cross section of a distal portion of a catheter, consistent with an embodiment of the present disclosure.

FIG. 3 illustrates another cross section of a distal portion of a catheter, consistent with an embodiment of the present disclosure.

FIG. 4 illustrates a sectional view of a catheter device, consistent with an embodiment of the present disclosure.

FIG. 5 illustrates a cross section of a spiral cut hypotube, consistent with an embodiment of the present disclosure.

FIG. 6 is a perspective view of the distal portion of the catheter.

FIG. 7 is an end view of the catheter showing dimensions of a certain embodiment.

FIG. 8 is an end view of the catheter.

### DETAILED DESCRIPTION OF THE PREFERRED EMBODIMENTS

In coronary heart disease the vasculature of the heart may have clot burden in the atherosclerotic vessels, which limit or restrict flow in these vessels. Traditionally, the physician would cross the lesion with a guidewire prior to advancing a Plain Old Balloon Angioplasty (POBA) to create a patent lumen (e.g., an inner open space or cavity of a tube having an internal diameter that is free from blockages or occlusion and capable of wire passage). The physician would then follow-up with the placement of a bare metal or drug eluting stent (DES), pinning the plaque and clot between the struts and the wall of the vessel. Applicants have recognized from clinical studies the improved patient outcomes when the soft clot burden or atherosclerotic debris in the coronary vessels is removed prior to stenting. In this regard, the concepts discussed herein disclose a novel and effective method and apparatus to remove this clot burden.

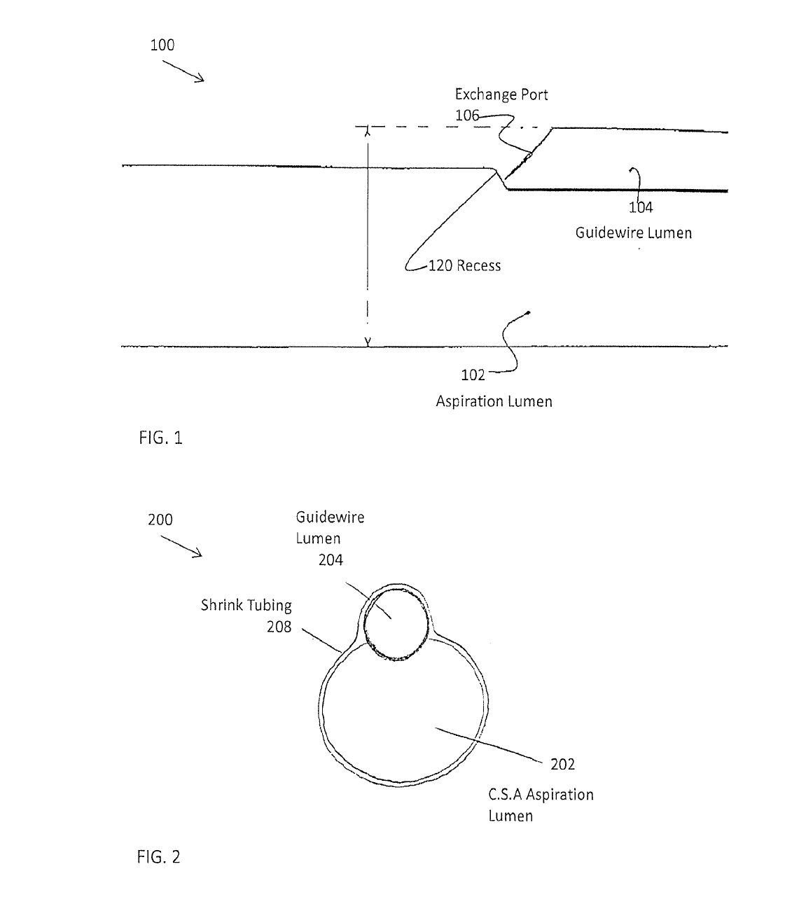

FIG. 1 illustrates a catheter, consistent with an embodiment of the present disclosure. Catheter 100 includes an aspiration lumen 102 and a guidewire lumen 104. The guidewire lumen 104 includes an exchange port 106. In one aspect of the present disclosure, a recess 120 is included in the aspiration lumen 102. A segment of the aspiration lumen 102 is substantially removed over the distal portion of the aspiration lumen to seat the guidewire lumen. A guidewire lumen may be a “Rapid Exchange” (Rx) lumen (e.g., a catheter where only the distal portion is delivered over the guide wire.) For example, the guide wire vents (e.g., exits) out through a side port and runs externally and parallel to the catheter proximal to this port. Thus, the guide wire lumen runs off center with respect to the aspiration lumen 102. Such configuration substantially improves (i.e., effectively maximizes) the aspiration lumen cross sectional area while keeping the overall profile of the assembly small. For example, the configuration discussed herein is smaller than commercial catheter designs (e.g. Medtronic Export Catheter). In one embodiment, the overall profile of the system is compatible with a guide catheter having ≦0.070″ internal diameter, which is representative of a 6F Guide catheter (i.e., the workhorse catheter for coronary intervention).

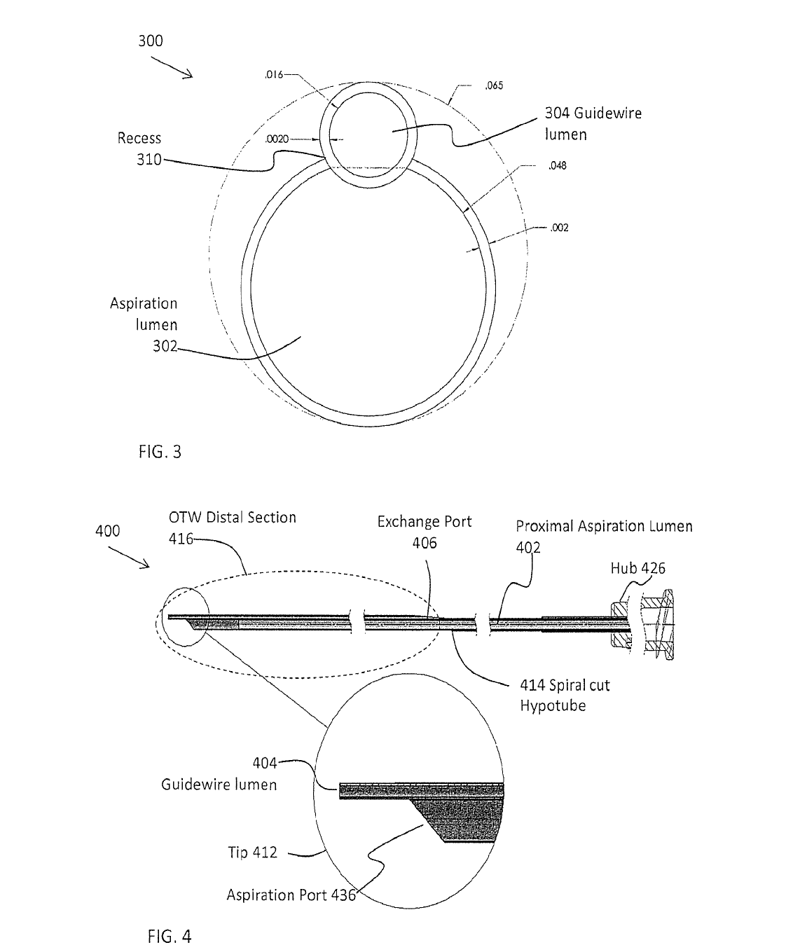

Reference now is made to FIGS. 2 and 3, which illustrate cross sections of a distal portion of a catheter, consistent with embodiments of the present disclosure. FIG. 2 illustrates a catheter 200 having an aspiration lumen 202, a guidewire lumen 204 on one side of the catheter 200, and shrink tubing 208 around the aspiration lumen 202 and guidewire lumen 204. By configuring the guidewire lumen at the side of the aspiration lumen 202, the predetermined cross sectional area (C.S.A) of the aspiration lumen 202 is improved.

FIG. 3 illustrates a cross section of a catheter 300 having several features similar to those discussed in the context of FIG. 2. By way of example, FIG. 3 provides dimensions of components that may be used in an exemplary catheter 300, consistent with an embodiment of the present disclosure. Unlike conventional configurations, where the distal end of the aspiration lumen is deformed to form a crescent shape cross sectional (e.g., in an effort to keep the outer diameter profile to a minimum), the embodiment of FIG. 3 achieves a similar result by replacing the portion of the aspiration luminal wall circumference with the wall of the guidewire lumen 304. Accordingly, there is minimal loss to the aspiration C.S.A. Put differently, the cross sectional area of the aspiration lumen is substantially improved (e.g., maximized).

FIG. 4 illustrates a sectional view of a catheter device 400, consistent with an embodiment of the present disclosure. In the example of FIG. 4, the catheter aspiration lumen 402 is segmented into 2 sections: (1) over the wire (OTW) distal section 416; and (2) the proximal section (e.g., aspiration lumen) 402. The distal OTW section 416 of the catheter 400 is defined by the guide wire lumen 404 that travels in line with the aspiration lumen 402. Thus, the distal OTW section 416 is the portion of the catheter that travels over a guidewire 404. The OTW section 416 is defined by an exchange port 406 along the shaft and the tip 412 at the end of the catheter device 400. For example, a guidewire passes through the chamfered aspiration lumen 402 at the exchange port 406 and the guidewire extends beyond the smaller wire lumen (i.e., guidewire lumen) at the distal end 416 (where both tubes terminate). Thus, the guidewire does not terminate at the end of the catheter but extends beyond it. During a procedure, the guidewire is positioned in the patient's vessel first and the aspiration lumen travels over the guidewire. In one embodiment, section 416 is the only portion of the catheter device 400 that encapsulates the guidewire.

In one implementation, OTW distal section 416 comprises a stainless steel hypotube 414. For example, the hypotube 414 may have a selectively cut laser pattern, which provides an optimal stiffness profile giving the user better control in delivery and support when positioned at the target treatment site.

Referring back to FIG. 3, in one example, a recess may be cut into the machine cut hypotube shaft over the OTW distal section 416. This recess 310 permits the guidewire lumen 304 to be seated. As discussed above, this section of the aspiration tubing wall is replaced by the guidewire tubing wall, when seated. Accordingly, the C.S.A of the aspirating lumen 302 is substantially preserved. That is because the guidewire lumen 304 does not obstruct the aspiration lumen 302 the way that prior art guidewire lumens do, as discussed above.

Referring back to FIG. 2, in one implementation, a shrink tube 208 comprising Pebax or Fluorinated Ethylene Propylene (FEP) polymer is placed over the hypotube (e.g., aspiration lumen (202)) and guidewire lumen 204 to join them and provide a fluid tight seal between the aspiration lumen 202 and guidewire wire lumen 204 of the catheter assembly 200. For example, the proximal aspiration lumen 402 (see FIG. 4), from the wire exchange port 406 to the hub 426, may comprise a laser cut tube having a material substantially similar to that of the hypotube 414.

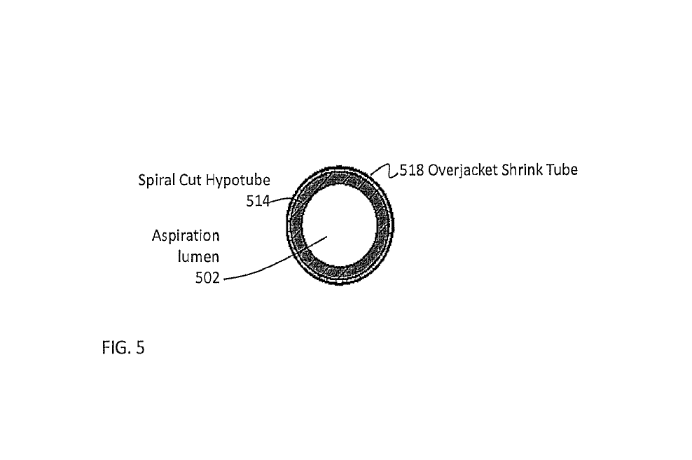

FIG. 5 illustrates a cross-sectional view of a spiral cut hypotube 514, consistent with an embodiment of the present disclosure. The proximal aspiration lumen 502 may be enclosed in a shrink tubing 518 or a polymer tube reinforced with a metal coil or braid with a hub attached to the proximal end. Such configuration provides a fluid tight seal up to the aspiration port 436, allowing a vacuum to be applied to remove material (e.g., thrombus &/or debris) into a lock syringe (not shown). For example, in one embodiment, the lock syringe may be an accessory used during the procedure and part of the device 400.

One factor that may limit the size of the C.S.A of the aspiration lumen 302 is the overall diameter profile restriction that may result from the stack-up of aspiration lumen 302 with the guidewire lumen 304 diameter in the distal end 416 of the assembly 400. In this regard, commercially available devices that are compatible with a 0.070″ catheter lumen or a 6F guide catheter are generally limited to an outer diameter profile of less than or equal to 0.067″.

By way of example, calculations are provided below for C.S.A's of a catheter, consistent with an embodiment of the present disclosure, and compared to the largest known commercially available technology compatible with a 6F Guide. The percentage gain in C.S.A as compared to a known commercial device is also calculated.

In commercial designs the aspiration lumen may have a diameter of approximately 0.043″. In this regard, the C.S.A=π·r̂2, where r=0.0215. Accordingly, the corresponding C.S.A=1.45×10⁻³ in².

In contrast, based on the concepts discussed herein, an exemplary aspiration lumen diameter may have a diameter of approximately 0.048″. In this regard, the C.S.A=π·r̂2, where r=0.0215. Accordingly, the corresponding C.S.A=1.81×10⁻³ in², which is substantially larger than the C.S.A calculated above for the prior art.

Subtracting the portion of the C.S.A that is replaced by the guidewire lumen (see FIG. 3), which may be 4.01×10⁻⁵ in², provides a C.S.A in the aspirating lumen of approx. 1.77×10⁻³ in². The value of 4.01×10⁻⁵ in² in this example may be a value provided when seating the guidewire lumen into the aspiration lumen. The C.S.A. of each lumen may be calculated an estimate made as to how much the small lumen removed from the large aspiration lumen.

Thus, the calculations above demonstrate by comparing the C.S.A. of 1.77×10-3 in² of the exemplary embodiment to a commercially available design of 1.45×10-3 in², that there is a 22% increase in cross sectional area while the overall outer diameter of the exemplary embodiment is approx. 0.067″ on the maximum O.D.

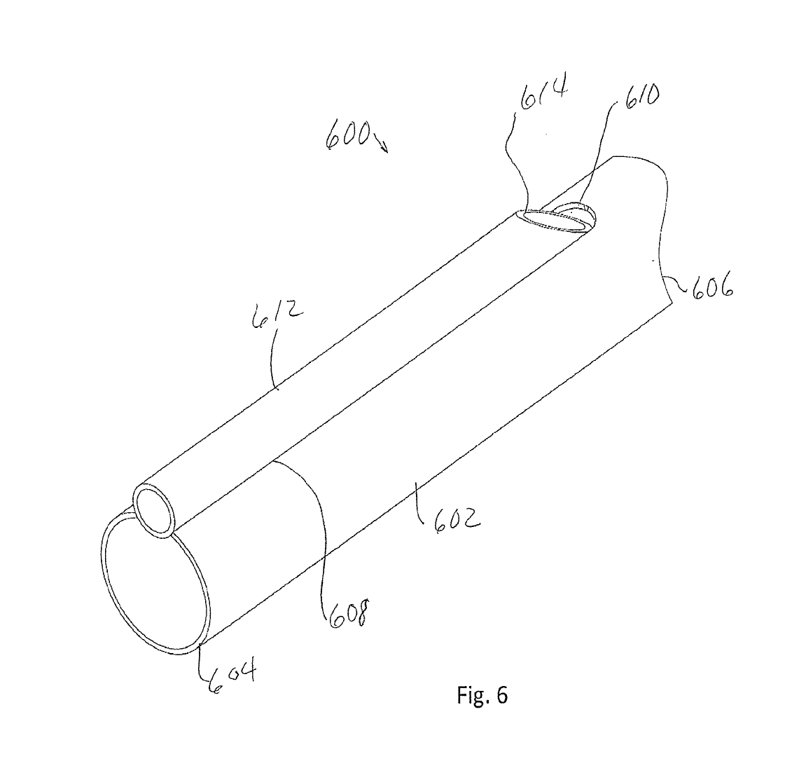

FIG. 6 is a perspective view of a portion of the catheter 600. The catheter includes an aspiration catheter 602 of a generally cylindrical, hollow overall shape. The aspiration catheter 602 has a free end 604 that is transverse to the length of the catheter 602. The catheter 602 extends a length greater than shown in the drawing, as indicated at 606. The aspiration catheter 602 has been shaped to have a recess 608 that extends from the free end 604 to a rounded or semicircular end 610 along a portion of the length of the aspiration catheter 602. The recess 608 forms an opening through the wall of the aspiration catheter 602. In the illustrated embodiment, the recess has a width that extends about over approximately 20 percent of the circumference of the aspiration catheter. Other widths, lengths, and shapes are possible for the recess 608.

A guidewire lumen 612 has a generally cylindrical, hollow overall shape and of a smaller diameter than the aspiration catheter 602. The guidewire lumen 612 is affixed in the recess 608 s as to extend parallel to the aspiration catheter 602 with a smaller portion of the guidewire lumen 612 extending into the recess 608 and a larger portion of the guidewire lumen 612 extending outside the aspiration catheter 602. The guidewire lumen 612 is affixed to the aspiration catheter 602 such as by welding or other means. The guidewire lumen 612 extends nearly the full length of the recess 608 from the free end 604 of the aspiration catheter 602 to near the rounded end 610 of the recess 608. The end of the guidewire lumen 612 near the rounded end 610 of the recess 608 has an angled exchange port 614.

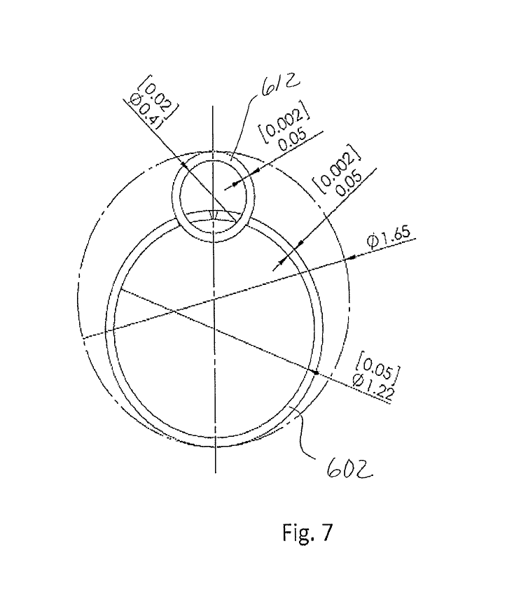

FIG. 7 shows the aspiration catheter 602 of FIG. 6 with the guidewire lumen 612 mounted thereto. In the illustrated example, the interior diameter of the aspiration catheter 602 is 1.22 mm, the thickness of the walls of the aspiration catheter is 0.05 mm, the interior diameter of the guidewire lumen is 0.41 mm and the wall thickness of the guidewire lumen is 0.05 mm. The entire assembly fits within a circle of diameter 1.65 mm.

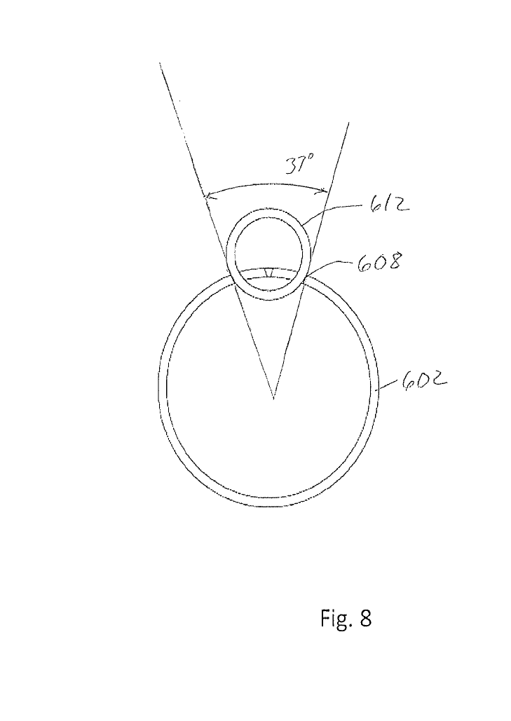

FIG. 8 shows the end view of the aspiration catheter 602 of FIGS. 6 and 7. The opening in the aspiration catheter that holds the guidewire lumen 612 is approximately 35 to 40 degrees or about 20 percent of the circumference of the aspiration catheter. In one example, the guidewire lumen 612 extends over about 37 degrees of the aspiration catheter 60.

The components, steps, features, objects, benefits and advantages that have been discussed are merely illustrative. None of them, or the discussions relating to them, is intended to limit the scope of protection in any way. Numerous other embodiments are also contemplated. These include embodiments that have fewer, additional, and/or different components, steps, features, objects, benefits and advantages. These also include embodiments in which the components and/or steps are arranged and/or ordered differently.

I claim:

1. An aspiration catheter comprising:
an aspiration lumen;
a guidewire lumen having an exchange port; and
a recess in the aspiration unit;
wherein the aspiration catheter lumen is divided into:
a proximal section;
a distal section having a distal opening; and
wherein the exchange port is configured between the distal segment and the proximal segment.

2. The aspiration catheter of claim 1, wherein the guidewire lumen is a “Rapid Exchange” lumen.

3. The aspiration catheter of claim 1, wherein the guidewire lumen is at the side of the aspiration lumen.

4. The aspiration catheter of claim 1, wherein a portion of an aspirational luminal wall is replaced with a wall of the guidewire lumen.

5. The aspiration catheter of claim 1, wherein distal section comprises a catheter that is delivered with the guide wire lumen through a central lumen.

6. The aspiration catheter of claim 1, wherein distal section comprises a stainless steel hypotube having a selectively cut laser pattern.

7. The aspiration catheter of claim 1, further comprising a shrink tube that joins the aspiration lumen and the guidewire lumen providing and is configured to provide a fluid tight seal between the aspiration lumen and the guidewire lumen.
